(19) United States
(12) Patent Application Publication
(10) Pub. No.: US 2022/0087833 A1
(43) Pub. Date: Mar. 24, 2022

(54) MAGNETICALLY COUPLED POWER SYSTEM FOR A POWERED MOBILITY ASSISTANCE DEVICE INCLUDING FAST SWAP BATTERY

(71) Applicant: Parker-Hannifin Corporation, Cleveland, OH (US)

(72) Inventor: Ryan J. FARRIS, Solon, OH (US)

(21) Appl. No.: 17/419,867

(22) PCT Filed: Jun. 4, 2019
(86) PCT No.: PCT/US2019/035286

§ 371 Date: Jun. 30, 2021

Related U.S. Application Data
(60) Provisional application No. 62/831,842, filed on Apr. 10, 2019.

(51) Int. Cl. A61F 2/60; A61F 5/01; A61H 1/02; B25J 9/00; A61F 2/70; B25J 19/00

(52) U.S. Cl. CPC A61F 2/60; A61F 5/0111; A61H 1/024; A61F 5/0123; A61F 2/70; B25J 19/005; B25J 9/0006

(57) ABSTRACT

A power system includes a battery assembly that has at least one battery cell for providing power; a battery receiver that removably receives the battery assembly; and a plurality of first connecting elements that are located in the battery assembly and a corresponding plurality of second connecting elements that are located in the battery receiver, wherein an attractive magnetic force between the plurality of first and second connecting elements aids in maintaining a physical connection between the battery assembly within the battery receiver. In addition, a connection between a pair of a first connecting element and a corresponding second connecting element further constitutes an electrical connection between the battery assembly and the battery receiver. The battery system may be incorporated into a mobility assistance device that includes a first limb component that includes the battery receiver and an actuator system that is powered when the battery assembly is connected to the battery receiver, and a second limb component, wherein a connection of the first and second limb components comprises a rotatable joint, and the actuator system rotates the second limb component relative to the first limb component at the joint.

### RELATED APPLICATIONS

This application claims the benefit of U.S. Provisional Application No. 62/831,842 filed Apr. 10, 2019, the contents of which are incorporated herein by reference.

### FIELD OF INVENTION

The present invention relates to powered mobility assistance devices, such as a powered leg brace or exoskeleton device, and more particularly to enhanced power systems including battery packs for powering such devices.

### BACKGROUND OF THE INVENTION

There are currently on the order of several hundred thousand spinal cord injured (SCI) individuals in the United States, with roughly 12,000 new injuries sustained each year at an average age of injury of 40.2 years. Of these, approximately 44% (approximately 5300 cases per year) result in paraplegia. One of the most significant impairments resulting from paraplegia is the loss of mobility, particularly given the relatively young age at which such injuries occur. Health conditions or ailments such as strokes, degenerative muscular and neurological diseases, as well as injuries also can result in substantially impaired mobility that may be short of full paralysis, but still relatively debilitating. Surveys of users with such impairments indicate that mobility concerns are among the most prevalent, and that chief among mobility desires is the ability to walk and stand. In addition to impaired mobility, the inability to stand and walk entails severe physiological effects, including muscular atrophy, loss of bone mineral content, frequent skin breakdown problems, increased incidence of urinary tract infection, muscle spasticity, impaired lymphatic and vascular circulation, impaired digestive operation, and reduced respiratory and cardiovascular capacities.

In an effort to restore at least some degree of legged mobility to individuals with paraplegia or significantly impaired mobility, the use of powered orthoses has been under development, which incorporate actuators and drive motors associated with a power supply to assist with locomotion. These powered orthoses have been shown to increase gait speed and decrease compensatory motions, relative to walking without powered assistance. The use of powered orthoses presents an opportunity for electronic control of the orthoses, for enhanced user mobility. An example of the current state of the art of exoskeleton devices is shown in Applicant's International Application Serial No. PCT/US2015/23624, entitled “Wearable Robotic Device,” filed Mar. 31, 2015.

Battery life is a common issue with powered orthoses and wearable robotic devices. Due to the desire to keep wearable robotic devices as small and light as possible, large battery packs are sometimes avoided in favor of smaller power options. In the case of powered orthotics, which require significant electrical energy to drive joint actuators and not simply sensor electronics, small battery packs may not provide adequate run time for the desired application. For instance, some commercially available exoskeleton devices may provide only two hours of run time on a battery charge. This may not be acceptable in some use cases. Two main options have been explored to address the challenge of battery life for wearable robotic devices. One option is to increase battery size and weight, which as referenced above is not desirable given the desire for smaller and lighter wearable robotics devices. Alternatively, therefore, attempts have been made to provide multiple, relatively small battery packs that can be readily transported and swapped as needed. Because users of wearable robotic devices such as exoskeleton devices and powered orthoses typically suffer from mobility and/or dexterity impairment, it remains a challenge to provide a suitable power system that incorporates small battery packs that can be swapped easily by users of wearable robotic devices and other powered orthoses.

### SUMMARY OF THE INVENTION

The present invention is directed to a power system that uses magnetic forces to connect a battery assembly and a battery receiver to provide for both physical and electrical connection between the battery assembly and the battery receiver. The power system, for example, may be used to power a powered mobility assistance devices, such as powered limb or gait orthoses or wearable robotic legged mobility devices or “exoskeletons,” and more particularly to a power system for mobility assistance devices that employs a removable battery assembly that can be readily transported and easily swapped, even by persons who may have significant physical impairments. The power system provides for interchangeable battery packs that may be swapped during use of a powered mobility assistance device, wherein a battery pack connection interface mechanically guides a battery pack into or onto a receiving unit on a device component, drawing the battery pack into a powered connection using a magnetic force which further secures the battery in place during use.

In exemplary embodiments, a power system for a powered mobility assistance device includes a battery assembly and a battery receiver incorporated as part of a component of the mobility assistance device, such as a leg component, wherein the battery receiver receives the battery assembly. The battery assembly may be configured as a battery back that is enclosed in a plastic housing, and the battery pack may contain any number of battery cells of any chemistry. For example, in one embodiment the battery pack includes six lithium ion cells of the 18650 variety. The battery assembly may include one more connecting elements, such as for example neodymium magnets, located within the battery assembly at an end of the battery pack near the electrical connection for electrically connecting the battery assembly to the battery receiver of the device (e.g. limb) component. In an exemplary embodiment, the one or more connecting elements may include a magnetic/electrical contact in which at least one of the magnet elements acts as both a physical connection using a magnetic force and an electrical connection.

The battery receiver of the device/limb component may be a metal or plastic structure designed to mechanically guide the battery assembly into alignment such that the battery pack electrical contacts correctly mate with receiver electrical contacts located within the battery receiver. The battery receiver also may include one or more connecting elements, such as neodymium magnets, which may be positioned with opposite polarity relative to corresponding magnet elements located within the batter assembly. In this manner, the opposing magnet elements of the battery assembly and the battery receiver attract each other, thus drawing the battery assembly into physical and electrical connection with the battery receiver. The connecting elements alternatively may be configured as opposing magnets and a ferrous material to provide the magnetic attraction. In an embodiment that uses an alternating polarized magnet installation, two mating magnet pairs may be provided in which one mating pair of battery assembly and battery receiver magnets has a battery assembly magnet with North facing the connection interface of the magnet pair, and one mating pair has a battery assembly magnet with South facing the connection interface of the magnet pair. The effect of this arrangement is that the battery assembly can only be installed facing in one direction, because when attempted to be installed backwards, the like poles of the opposing magnets will repel rather than attract in the backwards configuration. The connecting elements of the power system components also may include a magnetic/electrical contact in which at least one pair of the connecting elements acts as opposing magnetic/electrical contacts, which acts as both a physical connection using a magnetic force and an electrical connection.

With such configuration, the design of the power system enables fast battery assembly swapping and provides a secure connection between the battery assembly and the battery receiver. The connection may provide both a physical connection and an electrical connection through the magnetic attractive forces of opposing connecting elements of the battery assembly and the battery receiver, with the magnetic attractive force providing a secure connection in use but being readily overcome by manual action of the user for fast battery assembly swapping.

An aspect of the invention, therefore, is an enhanced power system that may be used, for example, in a mobility assistance device, wherein the power system incorporates a magnetically connected, removable battery assembly for fast swapping interchangeable battery assemblies with a battery receiver. In exemplary embodiments, the power system includes a battery assembly that has at least one battery cell for providing power to a powered device; a battery receiver that removably receives the battery assembly; and a plurality of first connecting elements that are located in the battery assembly and a corresponding plurality of second connecting elements that are located in the battery receiver, wherein an attractive magnetic force between the plurality of first and second connecting elements aids in maintaining a physical connection between the battery assembly within the battery receiver. In addition, a connection between a pair of a first connecting element and a corresponding second connecting element further constitutes an electrical connection between the battery assembly and the battery receiver. The power system may be incorporated into a mobility assistance device that includes a first limb component that includes the battery receiver and an actuator system that is powered when the battery assembly is connected to the battery receiver, and a second limb component, wherein a connection of the first and second limb components comprises a rotatable joint, and the actuator system rotates the second limb component relative to the first limb component at the joint.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto. Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a drawing depicting an isometric view of a mobility assistance device including an exemplary power system in accordance with embodiments of the present invention.

FIG. 2 is a drawing depicting a more close-up, isometric view of the exemplary power system of the mobility assistance device of FIG. 1, shown in a connected state.

FIG. 3 is a drawing depicting a more close-up, isometric view of the exemplary power system of the mobility assistance device of FIG. 1, shown in a disconnected state.

FIG. 4 is a drawing depicting an isometric and cross-section view of the exemplary power system in the disconnected state.

FIG. 5 is a drawing depicting an isometric and cross-section view of the exemplary power system in the connected state.

FIG. 6 is a drawing depicting an isometric view of the battery assembly component of the exemplary power system, with a portion of the battery assembly housing removed to illustrate the battery cells.

### DETAILED DESCRIPTION

Embodiments of the present invention will now be described with reference to the drawings, wherein like reference numerals are used to refer to like elements throughout. It will be understood that the figures are not necessarily to scale.

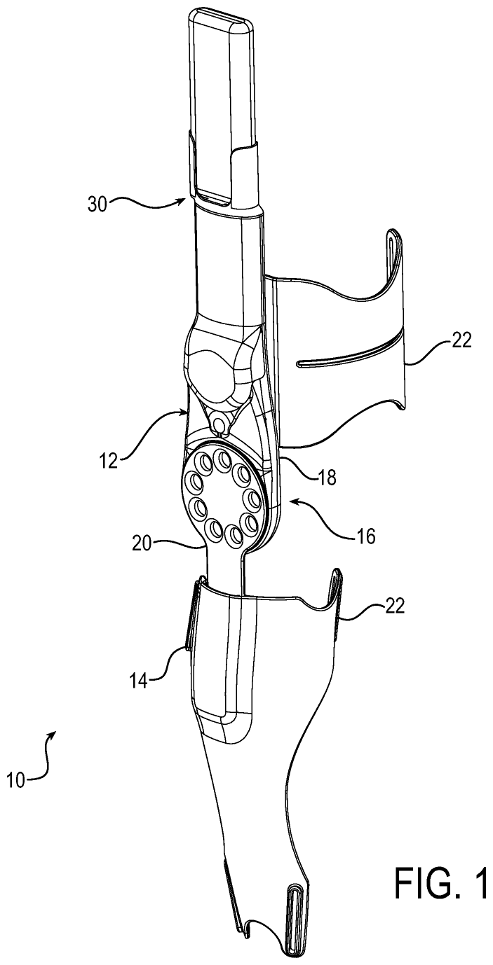

FIG. 1 is a drawing depicting an isometric view of a mobility assistance device 10 including an exemplary power system 30 in accordance with embodiments of the present invention. In this example, the mobility assistance device 10 is configured as a powered knee brace as an illustrative example of an application for the power system 30. It will be appreciated, however, that the powered knee brace device represents an example usage, and that the features of the power system are not limited to any particular configuration of a mobility assistance device. Rather, the principles of this invention may be applied generally to any suitable powered mobility assistance device or wearable robotic device. Such mobility assistance and wearable robotic devices include, for example, powered orthotic devices which aid in mobility for persons without use or limited use of a certain body portion, and powered prosthetic devices, which essentially provide an electro-mechanical replacement of a body part that is not present such as may be used by an amputee or a person congenitally missing a body portion. In addition, the mobility assistance device 10 is designed for mobility assistance associated with a single joint, e.g. a knee joint. Comparable principles may be applied to systems including multiple joints, including for example a bilateral exoskeleton device having two leg components that are attached to a hip component. As referenced above, such an exoskeleton device is described in Applicant's International Application Serial No. PCT/US2015/23624, entitled “Wearable Robotic Device,” filed 31 Mar. 2015.

As shown in the example of FIG. 1, the mobility assistance device 10, again configured as a powered knee brace, includes an upper leg or thigh component 12 that is attached to a lower leg or calf component 14. More generally, such components may be referred to as a first limb component 12 and a second limb component 14. The upper and lower leg (limb) components are attached to each other at a rotatable joint 16, whereby a first portion of the joint is provided as part of the upper leg component 12 and a second portion of the joint is provided as part of the lower leg component 14. In this example, the upper leg component 12 includes an actuator system 18 configured as an actuator cassette that incorporates a drive mechanism that includes an output stage that forms the first portion of the joint 16. The output stage of the actuator system 18 drives a joint connector 20 that forms the second portion of the joint 16 that is part of the lower leg component 14. During use, the drive mechanism imparts rotational motion of the lower leg component relative to the upper leg component to provide mobility assistance for such movements as standing, walking, sitting, and transitions between such movements. Any suitable configuration of the actuator system and joint may be employed in combination with the power system 30. Suitable examples of actuator systems, including actuator cassettes and related joint configurations, are described in Applicant's co-pending International Application Serial Nos. PCT/US2018/013990, PCT/US2018/013992, and PCT/US2018/013996, all filed on Jan. 17, 2018.

To attach the mobility assistance device 10 to the user, the device 10 can include attachment devices 22 for attachment of the device to the user via belts, loops, straps, or the like. Furthermore, for comfort of the user, attachment devices 22 can include padding disposed along any surface likely to come into contact with the user's limb.

The present invention pertains to a power system for mobility assistance devices that employs a removable battery assembly that can be readily transported and easily swapped, even by persons who may have significant physical impairments. The power system provides for interchangeable battery packs that may be swapped during use of a powered mobility assistance device, wherein a battery pack connection interface mechanically guides a battery pack into or onto a battery receiver unit located on a component of the mobility assistance device, drawing the battery pack into a powered connection using a magnetic force and securing the battery pack in place during use. The magnetic attractive force provides a secure connection in use but can be readily overcome by manual action of the user for fast battery assembly swapping. Although the power system is described in the context of use of driving a joint component in a wearable robotic device or powered mobility assistance device, the power system is not limited to such applications. Rather, the described power system is suitable for any field in which removable battery retention is desirable, and many such applications are suitable for the use of magnetic and magnetic/electrical connections of a battery or battery pack to a battery receiver as described herein.

An aspect of the invention, therefore, is an enhanced power system that may be used, for example, in a mobility assistance device, wherein the power system incorporates a magnetically connected, removable battery assembly for fast swapping interchangeable battery assemblies with a battery receiver. In exemplary embodiments, the power system includes a battery assembly that has at least one battery cell for providing power to a powered device; a battery receiver that removably receives the battery assembly; and a plurality of first connecting elements that are located in the battery assembly and a corresponding plurality of second connecting elements that are located in the battery receiver, wherein an attractive magnetic force between the plurality of first and second connecting elements aids in maintaining a physical connection between the battery assembly within the battery receiver. In addition, a connection between a pair of a first connecting element and a corresponding second connecting element further constitutes an electrical connection between the battery assembly and the battery receiver. The battery system may be incorporated into a mobility assistance device that includes a first limb component that includes the battery receiver and an actuator system that is powered when the battery assembly is connected to the battery receiver, and a second limb component, wherein a connection of the first and second limb components comprises a rotatable joint, and the actuator system rotates the second limb component relative to the first limb component at the joint.

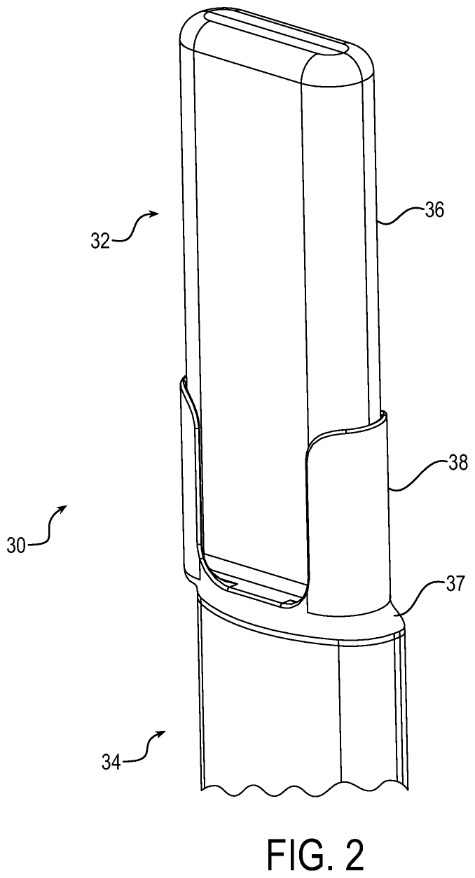

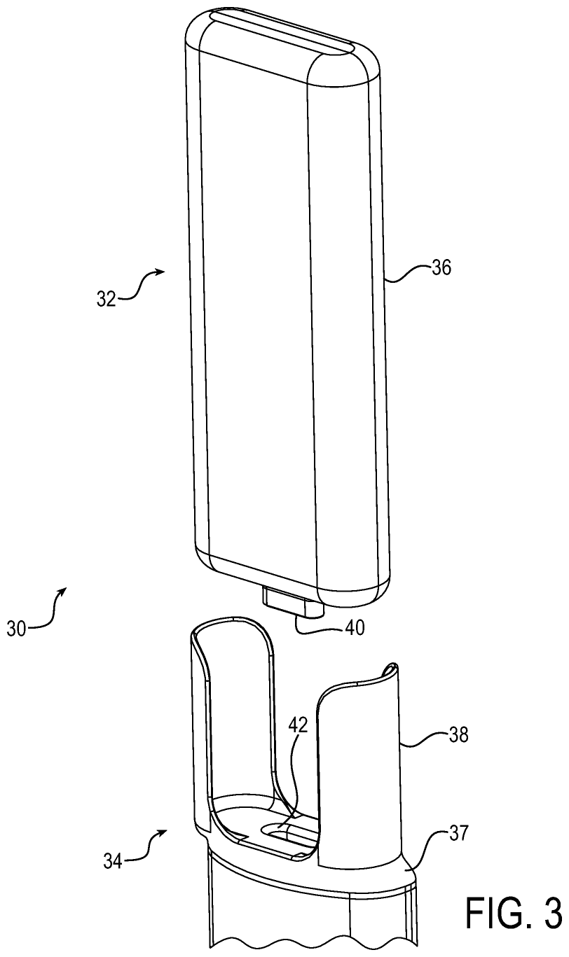

FIG. 2 is a drawing depicting a more close-up, isometric view of the exemplary power system 30 of the mobility assistance device 10 of FIG. 1, shown in a connected state. FIG. 3 is a drawing depicting a more close-up, isometric view of the exemplary power system 30 of the mobility assistance device 10 of FIG. 1, shown in a disconnected state. In exemplary embodiments, the power system 30 includes a battery assembly 32 and a device component (e.g., limb component 12) that includes a battery receiver 34 that receives battery assembly 32. The battery assembly 32 may be configured as a battery back that is enclosed in a housing 36, which typically is a rigid plastic housing, and the battery pack may contain any suitable number of individual battery cells of any chemistry. The battery receiver 34 may be incorporated as part of any suitable wearable robotic device, such as, in this particular example, the upper leg component 12 of the knee brace assistance device 10.

The battery receiver 34 may be a metal or rigid plastic structure. The battery receiver 34 further may include a receiver body 37 and guide structures 38 that extend from the receiver body 37, whereby the guide structures 38 operate to mechanically guide the battery assembly 32 into alignment such that the battery electrical contacts of the battery assembly 32 correctly mate with receiver electrical contacts located within the battery receiver 34. In this example, the guide structures 38 are configured as opposing guide fins that define a slotted structure that conforms to the shape of the battery assembly housing 36. In this manner, the battery assembly 32 can be readily located relative to the battery receiver 34 for easily connecting the two power system components.

As illustrated in the disconnected view of FIG. 3, the battery assembly 32 includes a battery connector 40, and the battery receiver 34 includes a receiver connector 42, which mate to form a physical and electrical connection between the battery assembly and battery receiver. Through such connection, power from the battery assembly may be applied via any suitable electrical pathways, such as by internal electrical wiring that passes through the first limb component 12, to the actuator system 18. In this example, the battery connector 40 is a male connector and the receiver connector 42 is a female connector, although such configuration may be reversed with the battery connector being a female connector and the receiver connector being a male connector. The opposing connectors first provide a secure mechanical connection, such as by a snap-in fit, that is readily connectable and disconnectable while maintaining a secured connection during use. The connection may be enhanced by incorporating a small degree of flexibility or spring-like action into the guide structures or fins 38. The guide structures 38 may be biased in an initial non-flexed state, and as the battery assembly 32 is inserted into the battery receiver 34, the battery housing 36 slightly spreads the guide structures 38 against the bias, which results in a tight snap fit of the guide structures 38 against the battery housing 36. The battery housing 36 and the guide structures 38 may have opposing rounded interfacing surfaces, as shown in FIGS. 2 and 3, to facilitate insertion of the battery assembly 32 into the battery receiver 34 and subsequent removal, which permits easy and fast swapping of interchangeable battery assemblies.

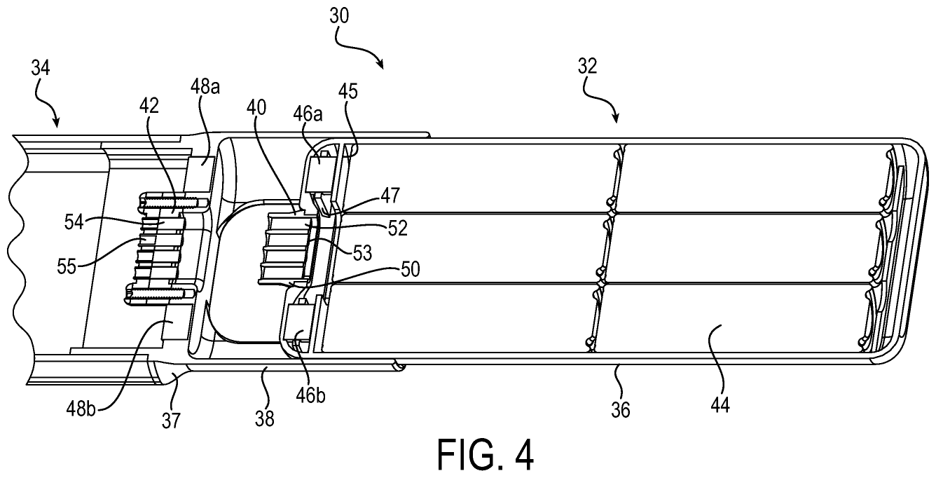

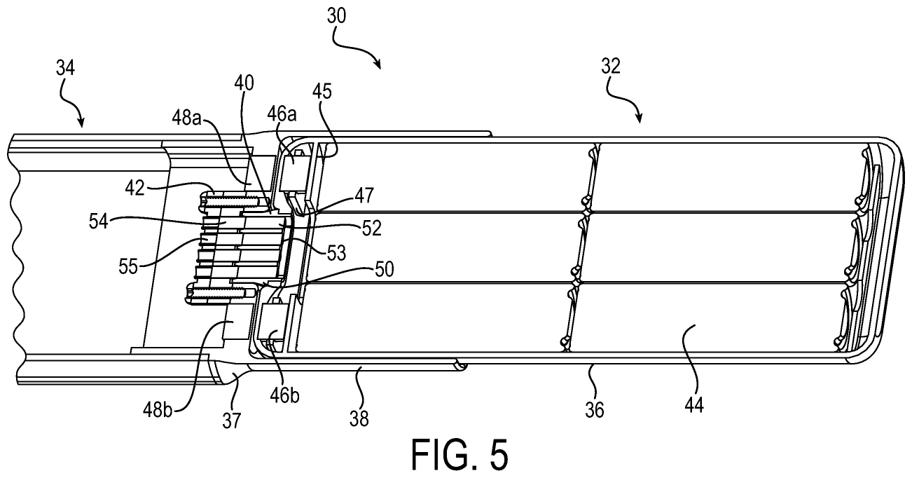

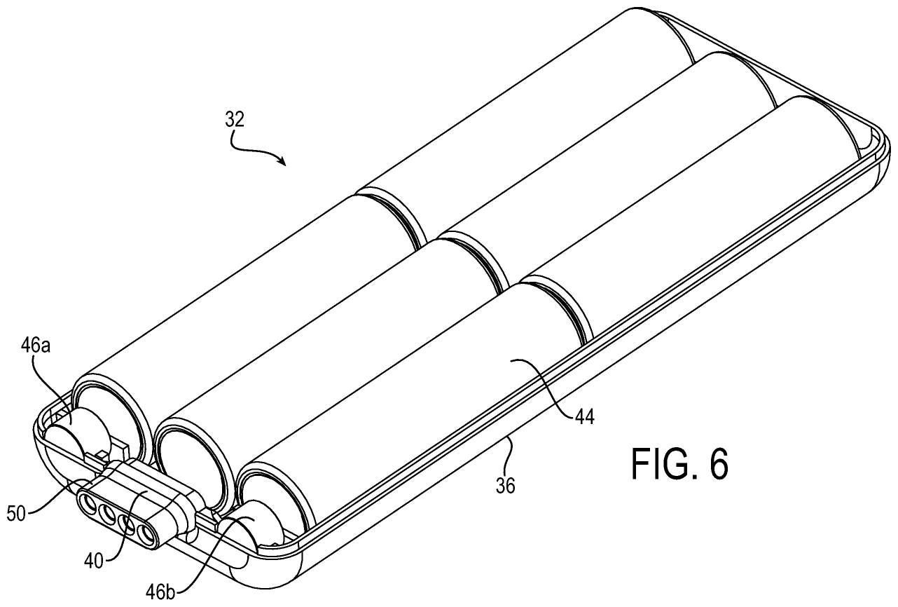

FIGS. 4-6 illustrate additional details of the power system 30, including additional features of the battery assembly 32 and the battery receiver 34. In particular, FIG. 4 is a drawing depicting an isometric and cross-section view of the exemplary power system 30 in the disconnected state. FIG. 5 is a drawing depicting an isometric and cross-section view of the exemplary power system 30 in the connected state. FIG. 6 is a drawing depicting an isometric view of the battery assembly component 32 of the exemplary power system 30 in isolation, with a portion of the battery assembly housing 36 removed.

As referenced above, and as illustrated in FIGS. 4-6, the battery assembly 32 may be configured as a battery back that includes one or more individual battery cells 44. Any suitable number of battery cells may be employed as appropriate for any particular wearable robotic device or other powered application, and the battery cells may be of any suitable chemistry. The selected number of battery cells may represent a balance between power needs and power longevity of a wearable robotic device versus size and weight constraints or limitations, as smaller size and less weight is more suitable for mobility. For example, in one exemplary embodiment as shown in the figures, the battery pack may include six lithium ion cells of the 18650 variety. In addition, the battery assembly and battery receiver may be standardized across numerous different types or configurations of wearable robotic devices. When such standardization is employed, battery assemblies may be used interchangeably with different types or configurations of wearable robotic devices.

For an enhanced secured connection, the battery assembly 32 may include one more battery assembly connecting elements 46 that aid in retaining the battery assembly to the battery receiver using a magnetic force, as further detailed below. Using magnetic forces, therefore, the connecting elements may encompass (1) actual magnets that can apply a magnetic force, and (2) a ferrous material that is attracted to a magnet by virtue of a magnetic force. In exemplary embodiments, therefore, the power system includes a battery assembly that has at least one battery cell for providing power, and a battery receiver that removably receives the battery assembly. A plurality of first connecting elements are located in the battery assembly and a corresponding plurality of second connecting elements are located in the battery receiver. The first and second connecting elements are selected to generate an attractive magnetic force between the plurality of first connecting elements and the plurality of second connecting elements to aid in maintaining a physical connection between the battery assembly within the battery receiver. To achieve the attractive magnetic force, opposing pairs of first and second connecting elements of the battery assembly and battery receiver include one magnet, and either a second magnet of opposing polarity or a connecting element made of a ferrous material that is attracted by an opposing magnet.

Accordingly, the first connecting elements 46 may be configured as actual magnets, such as for example neodymium magnets, or a ferrous material that is attracted to magnets contained in the battery receiver (which is described below). Any suitable number of first connecting elements may be employed, with two first connecting elements 46a and 46b being used in this example, and with one connecting element being provided on each side of the battery connector 40. Also in the depicted example, the first connecting elements 46 are housed in a compartment defined by housing extensions 45 that are formed as part of the battery housing 36. In this manner, the connecting elements 46 are isolated from the battery cells 44. The first connecting elements may be retained in place by retainers 47, which also may be extensions of the battery housing 36. Accordingly, the first connecting elements 46 are located within the battery assembly at a connection end adjacent to the battery connector 40, with the battery connector 40 electrically connecting the battery assembly 32 to the battery receiver 34.

The battery receiver 34 also may include one or more battery receiver or second connecting elements 48, which are positioned at the connection end respectively in opposing relation to the battery assembly first connecting elements 46. Again, similarly as above, the second connecting elements may be either actual magnets or made of a ferrous material that is attracted to magnet elements. It will be appreciated that with respect to each pair of opposing connecting elements 46 and 48, at least one of the opposing connecting elements is a magnet and the other of the opposing elements may be another magnet of opposite polarity or a ferrous material that is attracted to the opposing magnet. Accordingly, in this example there are two battery receiver second connecting elements 48a and 48b that are positioned oppositely relative to the battery assembly first connecting elements 46a and 46b. The second connecting elements 48 may be embedded within the material of the main body 37 of the battery receiver 34. The second connecting elements 48 may be made of a ferrous material that is attracted to the first connecting elements 46 that are actual magnets—or vice versa, the first connecting elements 46 may be made of a ferrous material that is attracted to the second connecting elements 48 that are actual magnets—thereby aiding the connection between the battery assembly and the battery receiver by an attractive magnetic force.

In a preferred embodiment, the first and second connecting elements 46 and 48 all are actual magnets, such as neodymium magnets, with the magnet elements 48 of the battery receiver being positioned with opposite polarity relative to the magnet elements 46 located within the battery assembly. In this manner, the opposing magnet elements of the battery assembly and the battery receiver attract each other, thus drawing the battery assembly into physical and electrical connection with the battery receiver with a stronger magnetic force. In the depicted example embodiment that uses such an alternating polarized magnet installation, two mating magnet pairs 46a/48a and 46b/48b may be provided in which one mating pair of battery assembly and battery receiver magnets has a battery assembly magnet with North facing the connection interface of the magnet pair, and one mating pair has a battery assembly magnet with South facing the connection interface of the magnet pair. The effect of this arrangement is that the battery assembly can only be installed within the battery receiver facing in one direction, because when attempted to be installed backwards, like poles of opposing magnets will repel each other rather than attract.

As referenced above, the battery assembly 32 includes a battery connector 40, and the battery receiver 34 includes a receiver connector 42, which mate to form a physical and electrical connection. The battery connector 40 includes a connector housing 50, which may be an extension of the battery housing 36. The connector housing 50 may house one or more magnet/electrical contacts 52 that are electrically connected with the battery cells 44 so as to transmit electrical power from the battery assembly 32. The receiver connector 42 of the battery receiver 34 may include one or more opposing electrical contacts 54 that become electrically connected to the magnet/electrical contacts 52 when the battery assembly and battery receiver are connected to each other.

In an exemplary embodiment, the magnet/electrical contacts 52 include a plurality of magnets, which as is typical of magnets are made of an electrically conductive material. Wires are soldered to the magnet/electrical contacts 52 at contact ends 53 so as to provide an electrical connection to the battery cells 44. The magnet/electrical contacts 52 may be generally cylindrical and are positioned for contacting the opposing electrical contacts 54 of the battery receiver 34. The electrical contacts 54 may be configured as pins made of a ferrous material, such as nickel-plated steel pins, that are respectively positioned to come in contact with the magnet/electrical contacts 52, as shown in the connected state of FIG. 5. The pins may be installed in a manner that permits the pins to float within the receiver connector 42, which ensures an effective electrical connection with the magnet/electrical contacts 52. Wires are soldered to the electrical contacts 54 at contact ends 55 to provide for downstream transmission of electrical power to the powered components of the mobility assistance device (or more generally to any powered device depending on the application). In another example, the electrical contacts 54 also may be electrically conductive magnets, having an opposite polarity relative to the magnet/electrical contacts 52. In the depicted example, a set of four opposing pairs of contacts 52/54 are provided, although any suitable number of contacts may be employed for a given application.

The additional magnetic coupling of the connectors 40 and 42 by the contacts 52 and 54 further aids in drawing the battery assembly into mechanical connection with the battery receiver. As referenced above, electrical wiring is soldered to the backsides of both magnet/electrical contacts 52 and 54. Accordingly, as a result of the joining of the contacts 52 and 54, an electrical connection via the opposing electrical elements or wiring is achieved, as well as a stable physical connection using the magnetic attractive force.

With such configuration, the design of the power system 30 enables fast battery assembly swapping while providing a secure connection between the battery assembly and the battery receiver during use. The connection may provide both a physical connection and an electrical connection through the opposing components of the battery assembly and the battery receiver, held together by the attractive magnetic force between opposing connecting elements. The magnetic attractive force provides a secure connection in use but can be readily overcome by manual action of the user for fast battery assembly swapping. In addition, because the connections include the magnetic coupling, optionally in combination with press fit mechanical connections of the housing components, no special tools or manipulations are required to connect and disconnect the battery assembly as needed to swap battery assemblies. The power system configuration, therefore, is suitable for effective use by persons who may have substantial physical or dexterity impairments to provide long-time powering of the wearable robotic device or other powered device for which fast swapping of battery packs is desirable.

An aspect of the invention, therefore, is an enhanced power system that may be used, for example, in a mobility assistance device, wherein the power system incorporates a magnetically connected, removable battery assembly for fast swapping interchangeable battery assemblies with a battery receiver. In exemplary embodiments, the power system includes a battery assembly that has at least one battery cell for providing power; a battery receiver that removably receives the battery assembly; and a plurality of first connecting elements that are located in the battery assembly and a corresponding plurality of second connecting elements that are located in the battery receiver. The first and second connecting elements are selected to generate an attractive magnetic force between the plurality of first connecting elements and the plurality of second connecting elements to aid in maintaining a physical connection between the battery assembly within the battery receiver. The power system may include one or more of the following features, either individually or in combination.

In an exemplary embodiment of the power system, the plurality of first connecting elements and the plurality of second connecting elements include corresponding first and second magnets of opposite polarity.

In an exemplary embodiment of the power system, a first mating pair of first and second magnets has a first magnet with North facing a connection interface of the first mating pair, and a second mating pair of first and second magnets has a first magnet with South facing a connection interface of the second mating pair.

In an exemplary embodiment of the power system, either of the plurality of first or second connecting elements comprises magnets, and the other of the plurality of first or second magnet elements comprises a ferrous material that is attracted to the magnets.

In an exemplary embodiment of the power system, at least a portion of the plurality of first and/or second connecting elements include neodymium magnets.

In an exemplary embodiment of the power system, the plurality of first connecting elements includes a magnet/electrical contact and the plurality of second connecting elements includes an opposing electrical contact, wherein a connection between the magnet/electrical contact and the opposing electrical contact further constitutes an electrical connection between the battery assembly and the battery receiver.

In an exemplary embodiment of the power system, the magnet/electrical contact includes a magnet and the opposing electrical contact is made of a ferrous material.

In an exemplary embodiment of the power system, the opposing electrical contact is a nickel-plated steel pin that contacts the magnet/electrical contact.

In an exemplary embodiment of the power system, the opposing electrical contact is a second magnet/electrical contact including a magnet having an opposite polarity relative to the magnet of the magnet/electrical contact.

In an exemplary embodiment of the power system, the battery assembly includes a battery connector that includes the magnet/electrical contact, and the battery receiver includes a receiver connector that includes the opposing electrical contact.

In an exemplary embodiment of the power system, the plurality of first and second connecting elements include a first pair of first and second connecting elements and a second pair of first and second connecting elements on opposite sides of the battery connector and the receiver connector.

In an exemplary embodiment of the power system, the battery assembly includes a battery housing the houses the at least one battery cell, and the battery housing includes a housing extension that defines a compartment that isolates the plurality of first connecting elements from the at least one battery cell.

In an exemplary embodiment of the power system, the battery housing further includes retainers that respectively retain the plurality of first connecting elements.

In an exemplary embodiment of the power system, the at least one battery cell comprises a plurality of lithium ion cells.

In an exemplary embodiment of the power system, the battery receiver includes a receiver body and guide structures that extend from the receiver body, and the guide structures guide the battery assembly into connection with the battery receiver.

In an exemplary embodiment of the power system, the guide structures comprise flexible fins that are biased in a non-flexed stated, wherein as the battery assembly is inserted into the battery receiver, the battery assembly spreads the fins to a flexed state to provide a tight snap-fit of the battery assembly within the battery receiver.

Another aspect of the invention is a powered mobility assistance device that includes the power system according to any of the embodiments, and a rotatable joint that is powered by the power system. In exemplary embodiments, the powered mobility assistance device includes a first limb component that includes the battery receiver and an actuator system that is powered when the battery assembly is connected to the battery receiver; and a second limb component, wherein a connection of the first and second limb components comprises the rotatable joint, and the actuator system rotates the second limb component relative to the first limb component at the joint.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A power system comprising:
a battery assembly that has at least one battery cell for providing power;
a battery receiver that removably receives the battery assembly; and
a plurality of first connecting elements that are located in the battery assembly and a corresponding plurality of second connecting elements that are located in the battery receiver;
wherein the first and second connecting elements are selected to generate an attractive magnetic force between the plurality of first connecting elements and the plurality of second connecting elements to aid in maintaining a physical connection between the battery assembly within the battery receiver.

2. The power system of claim 1, wherein the plurality of first connecting elements and the plurality of second connecting elements include corresponding first and second magnets of opposite polarity.

3. The power system of claim 2, wherein a first mating pair of first and second magnets has a first magnet with North facing a connection interface of the first mating pair, and a second mating pair of first and second magnets has a first magnet with South facing a connection interface of the second mating pair.

4. The power system of claim 1, wherein either of the plurality of first or second connecting elements comprises magnets, and the other of the plurality of first or second magnet elements comprises a ferrous material that is attracted to the magnets.

5. (canceled)

6. The power system of claim 1, wherein the plurality of first connecting elements includes a magnet/electrical contact and the plurality of second connecting elements includes an opposing electrical contact, wherein a connection between the magnet/electrical contact and the opposing electrical contact further constitutes an electrical connection between the battery assembly and the battery receiver.

7. The power system of claim 6, wherein the magnet/electrical contact includes a magnet and the opposing electrical contact is made of a ferrous material.

8. (canceled)

9. The power system of claim 6, wherein the opposing electrical contact is a second magnet/electrical contact including a magnet having an opposite polarity relative to the magnet of the magnet/electrical contact.

10. The power system of claim 6, wherein the battery assembly includes a battery connector that includes the magnet/electrical contact, and the battery receiver includes a receiver connector that includes the opposing electrical contact.

11. The power system of claim 10, wherein the plurality of first and second connecting elements include a first pair of first and second connecting elements and a second pair of first and second connecting elements on opposite sides of the battery connector and the receiver connector.

12. The power system of claim 1, wherein the battery assembly includes a battery housing the houses the at least one battery cell, and the battery housing includes a housing extension that defines a compartment that isolates the plurality of first connecting elements from the at least one battery cell.

13-14. (canceled)

15. The power system of claim 1, wherein the battery receiver includes a receiver body and guide structures that extend from the receiver body, and the guide structures guide the battery assembly into connection with the battery receiver.

16. The power system of claim 15, wherein the guide structures comprise flexible fins that are biased in a non-flexed stated, wherein as the battery assembly is inserted into the battery receiver, the battery assembly spreads the fins to a flexed state to provide a tight snap-fit of the battery assembly within the battery receiver.

17. A powered mobility assistance device comprising:
the power system according to claim 1; and
a rotatable joint that is powered by the power system.

18. The powered mobility assistance device of claim 17, further comprising:
a first limb component that includes the battery receiver and an actuator system that is powered when the battery assembly is connected to the battery receiver; and
a second limb component, wherein a connection of the first and second limb components comprises the rotatable joint, and the actuator system rotates the second limb component relative to the first limb component at the joint.

19. A powered mobility assistance device comprising:
a battery assembly that has at least one battery cell for powering the mobility assistance device;
a battery receiver that removably receives the battery assembly; and
a plurality of first connecting elements that are located in the battery assembly and a corresponding plurality of second connecting elements that are located in the battery receiver, wherein an attractive magnetic force between the plurality of first connecting elements and the plurality of second connecting elements aids in maintaining a physical connection between the battery assembly within the battery receiver; and
wherein a connection between a pair of a first connecting element and corresponding second connecting element further constitutes an electrical connection between the battery assembly and the battery receiver.

20. The powered mobility assistance device of claim 19, wherein the plurality of first connecting elements and the plurality of second connecting elements include corresponding first and second magnets of opposite polarity.

21. The powered mobility assistance device of claim 19, wherein either of the plurality of first or second connecting elements comprises magnets and the other of the plurality of first or second magnet elements comprises a ferrous material that is attracted to the magnets.

22. The powered mobility assistance device of claim 19, wherein the battery assembly includes a battery connector that includes the first connecting element that is part of the electrical connection, and the battery receiver includes a receiver connector that includes the second connecting element that is part of the electrical connection.

23. The powered mobility assistance device of claim 22, wherein the first connecting element that is part of the electrical connection includes a magnet and the second connecting element that is part of the electrical connection is made of a ferrous material.

24. (canceled)

25. The powered mobility assistance device of claim 19, further comprising:
a first limb component that includes the battery receiver and an actuator system that is powered when the battery assembly is connected to the battery receiver; and
a second limb component, wherein a connection of the first and second limb components comprises a rotatable joint, and the actuator system rotates the second limb component relative to the first limb component at the joint.
